(19) United States
(12) Patent Application Publication
(10) Pub. No.: US 2026/0201116 A1
(43) Pub. Date: Jul. 16, 2026

(54) PROTEIN ADSORPTION INHIBITOR

(71) Applicant: NOF CORPORATION, Tokyo (JP)

(72) Inventors: Koji SEKIGUCHI, Kawasaki (JP); Yumiko NISHIYAMA, Kawasaki (JP)

(73) Assignee: NOF CORPORATION, Tokyo (JP)

(21) Appl. No.: 19/442,063

(22) Filed: Jan. 7, 2026

(30) Foreign Application Priority Data
Jan. 10, 2025 (JP) 2025-004027

(51) Int. Cl. C08G 65/34

(52) U.S. Cl. CPC C08G 65/34

(57) ABSTRACT

A protein adsorption inhibitor containing an alkylene oxide derivative represented by the formula (1): Z—{O—[[(PO)ₐ/(EO)_{b}]-(AO)_{c}—H]}ₓ wherein symbols are as defined in the SPECIFICATION, wherein a 1% by mass aqueous solution of the alkylene oxide derivative has a clouding point of 0° C. or above and 30° C. or below is provided. The protein adsorption inhibitor can highly inhibit the adsorption of proteins onto the surfaces of immunoreaction container, measuring equipment, and the like, and can also reduce variability between measurements.

### TECHNICAL FIELD OF THE INVENTION

The present invention relates to a protein adsorption inhibitor.

### BACKGROUND OF THE INVENTION

In the fields of clinical testing and diagnostic agents used for the early detection of diseases, measurement methods utilizing immune reactions are widely used, and improving sensitivity is a major problem. One of the factors that influences the detection sensitivity when measuring using an immune reaction is the adsorption of the antibody or antigen to be the measurement target, or of labeled products thereof to be used in the measurement, to the surface of an immunoreaction container, measuring equipment, and the like. In addition, when a substance containing multiple types of biomolecules, such as serum, plasma, cell extract, urine, and the like is used as a sample, the measurement sensitivity may decrease due to the adsorption of proteins and the like in the aforementioned coexisting substance onto the surface of the immunoreaction container, measuring equipment, and the like.

In order to prevent a decrease in the measurement sensitivity, a method of inhibiting the adsorption of proteins involved in immune reactions by treating the surfaces of immunoreaction container, measuring equipment, and the like with a solution obtained by dissolving biological proteins, such as bovine serum albumin (BSA), casein, gelatin, and the like which are not involved in immune reactions, in a buffer, as shown in Johnson, D. A., Gautsch, J. W., Sportsman, J. R., and Elder, J. H. “Improved Technique Utilizing Nonfat Dry Milk for Analysis of Proteins and Nucleic Acids Transferred to Nitrocellulose”, Gene Anal. Technol., 1, 3-8, 1984, is generally used.

### SUMMARY OF THE INVENTION

However, the adsorption inhibitory effects of the adsorption inhibitors of the above-mentioned prior art are insufficient particularly in measurements that require high sensitivity. Furthermore, when the above-mentioned bio-derived adsorption inhibitors are used, a problem occurs in that the measurements vary greatly and it is difficult to obtain accurate measurement results with a small number of samples.

As adsorption inhibitors, JP 2010-169604 A proposes acrylamide derivatives, WO 2017/155019 proposes specific polyethylene glycol monomethacrylate polymers, and WO 2020/158787 proposes polypropylene glycol and the like. However, these adsorption inhibitors are still insufficient in suppressing the variability between measurements.

The problem to be solved by the present invention is to provide a protein adsorption inhibitor that can highly inhibit the adsorption of proteins such as antibodies and enzymes onto the surfaces of immunoreaction container, measuring equipment, and the like, and can also reduce variability between measurements.

The present inventors have conducted intensive studies and found that an alkylene oxide derivative having a specific clouding point and a specific structure can solve the above-mentioned problem, which resulted in the completion of the present invention. The present invention based on this finding provides the following.
[1]A protein adsorption inhibitor comprising an alkylene oxide derivative represented by the formula (1):

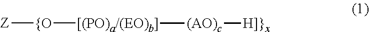

wherein
   x is a number between 1 and 4,
   Z is a residue having a structure obtained by removing hydroxy groups from a compound having 1 to 20 carbon atoms and hydroxy groups in the number of x,
   PO is an oxypropylene group,
   EO is an oxyethylene group,
   AO is an oxyalkylene group having 3 or 4 carbon atoms,
   a, b, and c respectively denote the number of the oxypropylene group, oxyethylene group, and oxyalkylene group, in which 1≤a≤50, 1≤b≤50, 0≤c≤50, 10≤(a+b+c)≤150, and 0.05≤b/(a+c)≤0.5, and
   [(PO) a/(EO)_{b}] denotes a polyoxyalkylene group in which PO in the number of a and EO in the number of b are bonded randomly and/or in blocks,
wherein a clouding point of a 1% by mass aqueous solution of the aforementioned alkylene oxide derivative is 0° C. or above and 30° C. or below.

### Effect of the Invention

By bringing an aqueous solution of the protein adsorption inhibitor of the present invention containing a specific alkylene oxide derivative in contact with the surface of a reaction container or the like at a temperature not less than the clouding point thereof, followed by removing same, adsorption of proteins such as antibody, enzyme, and the like to the aforementioned surface can be inhibited at a high level. Furthermore, using the above-mentioned aqueous solution, variability between measurements can also be reduced, and accurate measurement results can be obtained with a smaller number of samples.

### DETAILED DESCRIPTION OF THE INVENTION

The protein adsorption inhibitor of the present invention contains an alkylene oxide derivative represented by the formula (1):

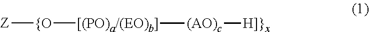

(sometimes to be abbreviated as “derivative (1)” in the present specification).

In the present specification, the “protein adsorption inhibitor” means an agent used to treat the surface of a reaction container and the like before contact of protein and the surface of the reaction container, in order to inhibit adsorption of the protein to the aforementioned surface. The aforementioned surface can be treated, for example, by contacting an aqueous solution of the protein adsorption inhibitor of the present invention containing derivative (1) with the aforementioned surface at a temperature of the clouding point thereof or above, and removing the aforementioned aqueous solution.

In the present specification, the “alkylene oxide” means a cyclic ether compound having a structure in which two carbon atoms are bonded by an oxygen atom. Examples of the alkylene oxide include ethylene oxide, propylene oxide, butylene oxide, tetrahydrofuran (tetramethylene oxide), and the like.

The protein adsorption inhibitor of the present invention preferably consists of derivative (1). Only one kind of derivative (1) may be used, or two or more kinds thereof may be used in combination. In addition, derivative (1) and other components may be used in combination as long as the effect of the present invention is not inhibited.

In the formula (1), x is a number which is 1 or more and 4 or less. From the viewpoint of protein adsorption inhibitory effect, x is preferably 1 or more and 3 or less, more preferably 1 or 2, further preferably 1.

In the formula (1), Z is a residue having a structure obtained by removing hydroxy groups from a compound having 1 to carbon atoms and hydroxy groups in the number of x. From the viewpoint of the protein adsorption inhibitory effect and inhibition of variability between measurements, the carbon number of Z is preferably 3 or more and 18 or less, more preferably 5 or more and 18 or less, further preferably 12 or more and 18 or less.

Examples of the compound having 1 or more and 20 or less carbon atoms and one hydroxy group include methanol, ethanol, propanol, butanol, lauryl alcohol, myristyl alcohol, cetyl alcohol, stearyl alcohol, oleyl alcohol, isodecyl alcohol, isostearyl alcohol, and the like. Examples of the compound having 1 or more and 20 or less carbon atoms and two hydroxy groups include ethylene glycol, propylene glycol, hexylene glycol, and the like. Examples of the compound having 1 or more and 20 or less carbon atoms and three hydroxy groups include glycerin, trimethylolpropane, and the like. Examples of the compound having 1 or more and 20 or less carbon atoms and four hydroxy groups include erythritol, pentaerythritol, sorbitan, diglycerol, alkylglycoside, and the like.

From the viewpoint of the inhibition of protein adsorption and inhibition of variability between measurements, Z is preferably a residue having a structure obtained by removing hydroxy group(s) from cetyl alcohol, stearyl alcohol, propylene glycol, glycerin, or pentaerythritol, more preferably a residue having a structure obtained by removing a hydroxy group from cetyl alcohol or stearyl alcohol.

In the formula (1), PO is an oxypropylene group, EO is an oxyethylene group, and AO is an oxyalkylene group having 3 or 4 carbon atoms.

In the formula (1), O—[(PO)ₐ/(EO)_{b}] means that the O at the left end in the aforementioned formula is bonded to a propylene group or an ethylene group in [(PO)ₐ/(EO)_{b}].

When c is 0, (AO)_{c} is absent, and [(PO)ₐ/(EO)_{b}]-(AO)_{c}—H in the formula (1) means that the H at the left end in the aforementioned formula is bonded to an oxy group in [(PO) a/(EO) b].

When c is not 0, [(PO)ₐ/(EO)_{b}]-(AO)_{c} in the formula (1) means that the oxy group in [(PO)ₐ/(EO)_{b}] is bonded to an alkylene group in (AO)_{c}, and (AO)_{c}—H in the formula (1) means that the H at the right end in the aforementioned formula is bonded to an oxy group in (AO)_{c}.

Examples of the AO include oxypropylene group, oxy(1-ethylethylene) group, and oxytetramethylene group. Among these, oxypropylene group and oxy(1-ethylethylene) group are preferred, and oxypropylene group is more preferred. When c is two or more, AO may be only one kind, or two or more kinds. When AO is two or more kinds, (AO)_{c} is a polyoxyalkylene group in which AO in the number of c is bonded randomly and/or in blocks. AO is preferably at least one selected from the group consisting of oxypropylene group and oxy(1-ethylethylene) group, more preferably oxypropylene group.

In the formula (1), a, b, and c respectively show the number of PO, EO, and AO. Each of a to c may be an average value, and therefore may be a decimal number.

The a is 1 or more and 50 or less (1≤a≤50), preferably 5 or more and 45 or less (5≤a≤45), more preferably 10 or more and or less (10≤a≤40), further preferably 20 or more and 40 or less (20≤a≤40). When a is 0, the protein adsorption inhibitory effect may decrease or the variability between measurements may increase, and when a is too large, the water solubility of derivative (1) may decrease.

The b is 1 or more and 50 or less (1≤b≤50), preferably 1 or more and 20 or less (1≤b≤20), more preferably 2 or more and or less (2≤b≤10), further preferably 3 or more and 5 or less (3≤b≤5). When b is 0, the water-solubility of derivative (1) may decrease, and when b is too large, the variability between measurements may increase.

The c is 0 or more and 50 or less (0≤c≤50), preferably 0 or more and 20 or less (0≤c≤20), more preferably 0 or more and or less (0≤c≤10), further preferably 0 (c=0). When c is too large, the protein adsorption inhibitory effect may increase.

The (a+b+c) is 10 or more (10≤(a+b+c)), preferably 15 or more (15≤(a+b+c)), more preferably 20 or more (20≤(a+b+c)). When (a+b+c) is less than 10, the protein adsorption inhibitory effect may decrease. The (a+b+c) is 150 or more ((a+b+c)≤150), preferably 100 or more ((a+b+c)≤100), more preferably 50 or more ((a+b+c)≤50). When (a+b+c) exceeds 150, the protein adsorption inhibitory effect may decrease.

The b/(a+c) is 0.05 or more and 0.5 or less (0.05≤b/(a+c)≤0.5), preferably 0.1 or more and 0.4 or less (0.1≤b/(a+c)≤0.4), more preferably 0.1 or more and 0.2 or less (0.1≤b/(a+c)≤0.2). When b/(a+c) is less than 0.05, the protein adsorption inhibitory effect may decrease, and when it exceeds 0.5, the variability between measurements may increase.

From the viewpoint of the protein adsorption inhibitory effect and inhibition of variability between measurements, x(a+b+c) showing the total number of oxyalkylene groups in one molecule of derivative (1) is preferably 10 or more and 200 or less (10≤x(a+b+c)≤200), more preferably 20 or more and 100 or less (20≤x(a+b+c)≤100), further preferably 25 or more and 45 or less (25≤x(a+b+c)≤45).

In the formula (1), [(PO)ₐ/(EO)_{b}] shows a polyoxyalkylene group in which PO in the number of a and EO in the number of b are bonded randomly and/or in blocks.

The order of binding of Z—{O and [(PO)ₐ/(EO)_{b}] in the formula (1) is not particularly limited, and (i) Z—{O and PO block in [(PO)ₐ/(EO)_{b}] may be bonded, or (ii) Z—{O and EO block in [(PO)ₐ/(EO)_{b}] may be bonded, or (iii) Z—{O and a part in which PO and EO in [(PO)ₐ/(EO)_{b}] are randomly bonded may be bonded. Preferably, Z—{O and PO block in [(PO)ₐ/(EO)_{b}] are bonded, or Z—{O and a part in which PO and EO in [(PO)ₐ/(EO)_{b}] are randomly bonded may be bonded, more preferably, Z-{Q and PO block in [(PO)ₐ/(EO)_{b}] are bonded.

The order of binding of [(PO)ₐ/(EO)_{b}] and (AO)_{c}—H in the formula (1) is not particularly limited, and (i) EO block in [(PO)ₐ/(EO)_{b}] and (AO)_{c}—H may be bonded, (ii) PO block in [(PO)ₐ/(EO)_{b}] and (AO)_{c}—H may be bonded, (iii) a part in which PO and EO in [(PO)ₐ/(EO)_{b}] are randomly bonded and (AO)_{c}—H may be bonded. Preferably, EO block in [(PO)ₐ/(EO)_{b}] and (AD)_{c}-H are bonded, or Z—{O and a part in which PO and EO in [(PO)ₐ/(EO)_{b}] are randomly bonded and (AO)_{c}—H are bonded, more preferably, EO block in [(PO)ₐ/(EO)_{b}] and (AD)_{c}-H are bonded.

The [(PO)ₐ/(EO)_{b}] is preferably a polyoxyalkylene group in which PO in the number of a and EO in the number of b are bonded randomly or in blocks, more preferably a polyoxyalkylene group in which PO in the number of a and EO in the number of b are bonded in blocks. Further preferably, [(PO)ₐ/(EO)_{b}] is a polyoxyalkylene group in which PO in the number of a and EO in the number of b are bonded in blocks, Z-{Q and (PO)ₐ block in [(PO)ₐ/(EO)_{b}] are bonded, and (EO)_{b} block in [(PO)ₐ/(EO)_{b}] and (AO)_{c}—H are bonded.

The clouding point of a 1% by mass aqueous solution of derivative (1) is 0° C. or above and 30° C. or below from the viewpoints of protein adsorption inhibitory effect and variability between measurements. The aforementioned clouding point is preferably 0° C. or above and 20° C. or below, further preferably 0° C. or above and 10° C. or below. Regarding the clouding point, JIS K 3211:1990 “Technical terms for surface active agents” describes, “the temperature at which an aqueous solution of a nonionic surfactant begins to become cloudy when the temperature is increased. Generally, the solution becomes cloudy and phase separation occurs”.

The clouding point of a 1% by mass aqueous solution of derivative (1) is a value measured by the following method. A 1% by mass aqueous solution of derivative (1) is placed in a test tube to a height of about 40 mm, and a thermometer is placed inside. The solution is heated to a temperature about 2 to 3° C. higher than the temperature at which clouding occurs, while stirring well with the thermometer, and then air-cooled while stirring well again. The temperature at which the solution has become transparent is taken as the clouding point. When the aqueous solution is cloudy at room temperature, the solution is cooled while stirring thoroughly until it becomes transparent, then gradually heated again while stirring thoroughly until it reaches a temperature at which the solution becomes cloudy, and then gradually cooled while stirring. The temperature at which it becomes transparent is taken as the clouding point.

The derivative (1) can be produced by a known method. When [(PO)ₐ/(EO)_{b}] is a polyoxyalkylene group in which PO in the number of a and EO in the number of b are bonded in blocks, derivative (1) can be obtained, for example, by subjecting either propylene oxide or ethylene oxide to addition polymerization to an alcohol corresponding to Z in the presence of an alkaline catalyst at a temperature of 50° C. or above and 160° C. or below and a pressure of 0.5 MPa (gauge pressure) or less, performing addition polymerization of the remaining one and then addition polymerization of an alkylene oxide having 3 or 4 carbon atoms, as necessary, then neutralizing the reaction mixture with an acid such as hydrochloric acid, phosphoric acid, acetic acid, and the like, and removing water and neutralized salts. When [(PO)ₐ/(EO)_{b}] is a polyoxyalkylene group in which PO in the number of a and EO in the number of b are bonded randomly, derivative (1) can be obtained, for example, by the same method as above except that a mixture of propylene oxide and ethylene oxide is subjected to addition polymerization to an alcohol corresponding to Z.

As described above, the treatment of a surface of a reaction container or the like for inhibiting protein adsorption on the aforementioned surface can be performed by using an aqueous solution of the protein adsorption inhibitor of the present invention containing derivative (1) (hereinafter sometimes to be abbreviated as “aqueous inhibitor solution”). From the viewpoint of the protein adsorption inhibitory effect and inhibition of variability between measurements, the concentration of derivative (1) in the aqueous inhibitor solution is preferably 0.05% by mass or more and 10% by mass or less, more preferably 0.1% by mass or more and 5.0% by mass or less, further preferably 0.2% by mass or more and 1.0% by mass or less.

Examples of the solvent for preparing the aqueous inhibitor solution include water (e.g., pure water, ion exchange water), physiological saline, and buffers used in immunological measurement methods, and the like. Examples of the buffer include phosphate buffer (e.g., Dulbecco phosphate buffer), acetate buffer, carbonate buffer, citrate buffer, Tris buffer, HEPES buffer, and the like.

The aqueous inhibitor solution may contain components other than derivative (1) as long as the effects of the present invention are not impaired. Examples of such other component include salts (e.g., sodium chloride contained in the aforementioned physiological saline) and buffering agents (e.g., buffering agent contained in the aforementioned buffer).

### EXAMPLES

The present invention is specifically described below by referring to Synthetic Example and Experimental Example. The examples are mere exemplifications and do not limit the present invention in any way.

### Synthetic Example 1: Synthesis of Alkylene Oxide Derivative 1

Stearyl alcohol (100 g) and potassium hydroxide (3.2 g) as a catalyst were charged into an autoclave. The air in the autoclave was replaced with dry nitrogen, and the catalyst was dissolved at 140° C. with stirring. Successively, propylene oxide (854 g) was added dropwise at 120° C. and 0.2 to 0.5 MPa (gauge pressure) using a dropping device, and the mixture was stirred at 120° C. for 3 hr. Then, ethylene oxide (85 g) was added dropwise at 120° C. and 0.2 to 0.5 MPa (gauge pressure) using a dropping device, and the mixture was stirred at 120° C. for 2 hr. The reaction mixture was then removed from the autoclave and neutralized with hydrochloric acid to adjust the pH to 6-7. The reaction mixture was then treated at −0.095 MPa (gauge pressure) and 100° C. for 1 hr to remove moisture. The resulting mixture was filtered to remove any salts formed during the treatment to obtain alkylene oxide derivative 1 (hereinafter referred to as “derivative 1”). The number average molecular weights of the polyoxyethylene and polyoxypropylene groups were calculated from the hydroxyl value determined by hydroxyl value measurement in accordance with JIS K1557-1, and the values of a and b were calculated from these number average molecular weights.

### Synthetic Examples 2 to 11: Synthesis of Alkylene Oxide Derivatives 2 to 11

Alkylene oxide derivatives 2 to 11 (hereinafter respectively referred to as “derivatives 2 to 11”) were obtained using a method similar to that used in Synthetic Example 1.

In Synthetic Example 2 (synthesis of derivative 2) and Synthetic Example 7 (synthesis of derivative 7), a mixture of propylene oxide and ethylene oxide was added to the starting material corresponding to Z to form [(PO)ₐ/(EO)_{b}]. In Synthetic Example 5 (synthesis of derivative 5) and Synthetic Example 6 (synthesis of derivative 6), an alkylene oxide corresponding to AO was added after the formation of [(PO)ₐ/(EO)_{b}]. In Synthetic Example 8 (synthesis of derivative 8), only propylene oxide was added to the starting material corresponding to Z. In Synthetic Example 10 (synthesis of derivative 10), only ethylene oxide was added to the starting material corresponding to Z.

The starting material corresponding to Z, the alkylene oxide corresponding to AO used in Synthetic Example 5 or 6, AO in derivative 5 or 6, the values of a, b, and c in the formula (1), and the values of (a+b+c), b/(a+c), and x(a+b+c) calculated from these values are shown in Tables 1 and 2. In addition, the clouding points of 1% by mass aqueous solutions of alkylene oxide derivatives 1 to 10 are also shown in Tables 1 and 2. Derivatives 1 to 6 are protein adsorption inhibitors of Examples, and derivatives 7 to 11 are protein adsorption inhibitors of Comparative Examples.

TABLE 1

| | Synthetic | Synthetic | Synthetic | Synthetic | Synthetic | Synthetic |
|---|---|---|---|---|---|---|
| | Example 1 | Example 2 | Example 3 | Example 4 | Example 5 | Example 6 |
| | derivative 1 | derivative 2 | derivative 3 | derivative 4 | derivative 5 | derivative 6 |
| starting material | stearyl | cetyl | propylene | penta- | penta- | glycerin |
| corresponding | alcohol | alcohol | glycol | erythritol | erythritol | |
| to Z | | | | | | |
| carbon number | 18 | 16 | 3 | 5 | 5 | 3 |
| of Z | | | | | | |
| x | 1 | 1 | 2 | 4 | 4 | 3 |
| [(PO)ₐ/(EO)_{b}] | block | random | block | block | block | block |
| binding form | | | | | | |
| alkylene oxide | — | — | — | — | propylene | butylene |
| corresponding | | | | | oxide | oxide |
| to AO | | | | | | |
| AO | — | — | — | — | oxypropylene | oxy (1- |
| | | | | | group | ethylethylene) |
| | | | | | | group |
| a | 38 | 24 | 18 | 16 | 5 | 35 |
| b | 5 | 3 | 3 | 1 | 5 | 20 |
| c | 0 | 0 | 0 | 0 | 15 | 8 |
| (a + b + c) | 43 | 27 | 21 | 17 | 25 | 63 |
| b/(a + c) | 0.13 | 0.13 | 0.17 | 0.06 | 0.25 | 0.47 |
| x(a + b + c) | 43 | 27 | 42 | 68 | 100 | 189 |
| clouding | 3 | 5 | 20 | 25 | 15 | 6 |
| point (° C.) | | | | | | |

TABLE 2

| | Syn- | Syn- | Syn- | Syn- | Syn- |
|---|---|---|---|---|---|
| | thetic | thetic | thetic | thetic | thetic |
| | Exam- | Exam- | Exam- | Exam- | Exam- |
| | ple 7 | ple 8 | ple 9 | ple 10 | ple 11 |
| | deriv- | deriv- | deriv- | deriv- | deriv- |
| | ative 7 | ative 8 | ative 9 | ative 10 | ative 11 |
| starting material | butanol | butanol | glycerin | ethanol | glycerin |
| corresponding | | | | | |
| to Z | | | | | |
| carbon number | 4 | 4 | 3 | 2 | 3 |
| of Z | | | | | |
| x | 1 | 1 | 3 | 1 | 3 |
| [(PO)ₐ/(EO)_{b}] | random | — | block | — | random |
| binding form | | | | | |
| alkylene oxide | — | — | — | — | — |
| corresponding | | | | | |
| to AO | | | | | |
| AO | — | — | — | — | — |
| a | 38 | 40 | 75 | 0 | 9 |
| b | 37 | 0 | 3 | 7 | 4 |
| c | 0 | 0 | 0 | 0 | 0 |
| (a + b + c) | 75 | 40 | 78 | 7 | 13 |
| b/(a + c) | 0.97 | — | 0.04 | — | 0.44 |
| x(a + b + c) | 75 | 40 | 234 | 7 | 39 |
| clouding | 48 | 0 or less | 12 | 80 or more | 38 |
| point (° C.) | | | | | |

### Synthetic Example 12: Synthesis of Methoxydiethylene Glycol Monoacrylate Polymer 12

Methoxydiethylene glycol monoacrylate (20 g) as a monomer, azobisisobutyronitrile (10 mg) as a polymerization initiator, and dioxane (60 g) were added to a four-necked flask, and the monomer and polymerization initiator were dissolved in dioxane. The polymerization reaction was performed at 70° C. for 8 hr. After completion of the reaction, the reaction mixture was diluted and washed with hexane, and the resulting solid was collected by filtration. The recovered solid was redissolved in 70 mL of tetrahydrofuran, and the resulting solution was again diluted and washed with hexane. The resulting solid was collected by filtration and dried under reduced pressure to obtain methoxydiethylene glycol monoacrylate polymer 12 (hereinafter referred to as “Polymer 12”). Analysis by GPC revealed that the number average molecular weight (Mn) of Polymer 12 was 18000. The clouding point of a 1% by mass aqueous solution of Polymer 12 was 41° C. This Polymer 12 is the protein adsorption inhibitor of Comparative Example.

### Experimental Example 1

### <Preparation of Aqueous Solutions of Protein Adsorption Inhibitor>

Derivatives 1 to 11 and polymer 12 obtained in Synthetic Examples 1 to 12 were each dissolved in Dulbecco phosphate buffer (manufactured by Sigma-Aldrich, hereafter abbreviated as “DPBS”) to a concentration of 0.5% by mass to prepare aqueous Solutions of Protein Adsorption Inhibitors.

<Evaluation of protein adsorption inhibitory effect>

The protein adsorption inhibitory effect was evaluated by the following method using the obtained aqueous solutions of the protein adsorption inhibitors.

An aqueous solution of the protein adsorption inhibitor was dispensed into a MaxiSoap plate (polystyrene plate manufactured by Thermofisher Scientific) at room temperature at 200 μL/well and allowed to stand at 37° C. for 1 hr. Thereafter, the aforementioned solution was completely removed using an aspirator, and DPBS containing 0.05% by mass of Tween 20 (hereinafter referred to as “DPBS-T”) was dispensed at 200 μL/well, and DPBS-T was then completely removed using an aspirator. The DPBS-T dispensing and removal procedure was repeated a total of three times.

Successively, POD-IgG (peroxidase-labeled immunoglobulin G, manufactured by Biorad) diluted 5,000-fold with DPBS was dispensed at 100 μL/well and allowed to stand at room temperature for 1 hr. Thereafter, DPBS-T was dispensed at 200 μL/well, and the aforementioned solution (mixture of POD-IgG diluted 5,000-fold with DPBS and DPBS-T) was completely removed using an aspirator. The DPBS-T dispensing and removal procedure was repeated a total of three times to wash the plate surface.

After washing, a color-developing solution prepared using TMB Microwell Peroxidase Substrate (manufactured by KPL) was added at 100 μL/well, and the enzyme (peroxidase) bound to the protein (immunoglobulin G) adsorbed to the MaxiSoap plate was reacted with TMB (tetramethylbenzidine) in the color-developing solution for 10 min at room temperature.

The color-developing reaction was stopped by adding 1 mol/L sulfuric acid solution at 50 μL/well, and the absorbance at 450 nm was measured. Smaller absorbance indicates greater inhibition of protein adsorption to the MaxiSoap plate. Infinite 200 PRO M-Plex (manufactured by TECAN) was used to measure absorbance.

Absorbance at 450 nm was measured in the same manner as above, except that bovine serum albumin (BSA) was used as the protein adsorption inhibitor. The absorbance obtained using BSA was normalized to 100, and the relative absorbance values obtained using each protein adsorption inhibitor are shown in Tables 3 and 4. A lower relative value mentioned above indicates greater inhibition of protein adsorption to the MaxiSoap plate. When the aforementioned relative value was less than 10, the protein adsorption inhibitory effect was evaluated as ⊙, when the aforementioned relative value was less than 100, the protein adsorption inhibitory effect was evaluated as ◯, and when the aforementioned relative value was 100 or greater, the protein adsorption inhibitory effect was evaluated as x. The evaluation results are shown in Tables 3 and 4. Protein adsorption inhibitors rated as ⊙ or ◯ for protein adsorption inhibitory effect were judged to be acceptable.

### <Evaluation of Variability Between Measurements>

To evaluate variability between measurements, the above-mentioned <Evaluation of protein adsorption inhibitory effect> was repeated 10 times, and the standard deviation of the absorbance measured 10 times was divided by the average absorbance measured 10 times to calculate the coefficient of variation (coefficient of variation=standard deviation of 10 absorbances/average of 10 absorbances).

Coefficient of variation of absorbance was calculated in the same manner as above, except that bovine serum albumin (BSA) was used as the protein adsorption inhibitor. With respect to variability between measurements, the proportion (%) of the coefficient of variation of the absorbance obtained using each protein adsorption inhibitor to the coefficient of variation of the absorbance obtained using BSA was calculated (=100×coefficient of variation of the absorbance obtained using each protein adsorption inhibitor/coefficient of variation of the absorbance obtained using BSA). The proportion of the coefficients of variation of absorbance are shown in Tables 3 and 4. A lower proportion mentioned above indicates greater inhibition of variability between measurements. When the aforementioned proportion was less than 50%, the variability between measurements was evaluated as ⊙, when the aforementioned relative value was 50% or more and less than 80%, the variability between measurements was evaluated as ◯, when the aforementioned relative value was 80% or more and less than 100%, the variability between measurements was evaluated as Δ, and when the aforementioned relative value was 100% or more, the variability between measurements was evaluated as x. The evaluation results are shown in Tables 3 and 4. Protein adsorption inhibitors rated as ⊙ or ◯ for variability between measurements were judged to be acceptable.

TABLE 3

| | protein adsorption inhibitor | | | | | |
|---|---|---|---|---|---|---|
| | derivative | derivative | derivative | derivative | derivative | derivative |
| | 1 | 2 | 3 | 4 | 5 | 6 |
| absorbance | 1.3 | 8 | 76 | 29 | 89 | 91 |
| relative value | | | | | | |
| protein adsorption | ⊙ | ⊙ | ◯ | ◯ | ◯ | ◯ |
| inhibitory effect | | | | | | |
| proportion (%) of | 20 | 36 | 31 | 60 | 73 | 60 |
| coefficient of | | | | | | |
| variation of absorbance | | | | | | |
| variability between | ⊙ | ⊙ | ⊙ | ◯ | ◯ | ◯ |
| measurements | | | | | | |

TABLE 4

| | protein adsorption inhibitor | | | | | |
|---|---|---|---|---|---|---|
| | derivative | derivative | derivative | derivative | derivative | derivative |
| | 7 | 8 | 9 | 10 | 11 | 12 |
| absorbance | 353 | 618 | 241 | 921 | 543 | 32 |
| relative value | | | | | | |
| protein adsorption | X | X | X | X | X | ◯ |
| inhibitory effect | | | | | | |
| proportion (%) of | 118 | 91 | 83 | 133 | 105 | 124 |
| coefficient of | | | | | | |
| variation of absorbance | | | | | | |
| Variability between | X | Δ | Δ | X | X | X |
| measurements | | | | | | |

The protein adsorption inhibitors of Examples (derivatives 1 to 6) were superior in the protein adsorption inhibitory effect, and also exhibited less variability between measurements.

In contrast, the protein adsorption inhibitors of Comparative Examples (derivatives 7 to 11 and polymer 12) were inferior in either or both of the protein adsorption inhibitory effects and variability between measurements.

### INDUSTRIAL APPLICABILITY

The protein adsorption inhibitor of the present invention can highly inhibit non-specific adsorption of proteins such as antibodies and enzymes onto the surfaces of immunoreaction container, measuring equipment, and the like, and can further reduce variability between measurements. Therefore, the protein adsorption inhibitor of the present invention is useful for in vitro diagnostics and the like.

This application is based on a patent application No. 2025-4027 filed in Japan, the contents of which are encompassed in full herein. In addition, the contents disclosed in any publication cited herein, including patents and patent applications, are hereby incorporated in their entireties by reference, to the extent that they have been disclosed herein.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIM.”

1. A protein adsorption inhibitor comprising an alkylene oxide derivative represented by the formula (1): 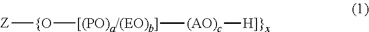
wherein
x is a number between 1 and 4,
Z is a residue having a structure obtained by removing hydroxy groups from a compound having 1 to 20 carbon atoms and hydroxy groups in the number of x,
PO is an oxypropylene group,
EO is an oxyethylene group,
AO is an oxyalkylene group having 3 or 4 carbon atoms,
a, b, and c respectively denote the number of the oxypropylene group, oxyethylene group, and oxyalkylene group, in which 1≤a≤50, 1≤b≤50, 0≤c≤50, 10≤(a+b+c)≤150, and 0.05≤b/(a+c)≤0.5, and
[(PO)ₐ/(EO)_{b}] denotes a polyoxyalkylene group in which PO in the number of a and EO in the number of b are bonded randomly and/or in blocks,
wherein a clouding point of a 1% by mass aqueous solution of the alkylene oxide derivative is 0° C. or above and 30° C. or below.
